(19) United States
(12) Patent Application Publication
(10) Pub. No.: US 2026/0248567 A1
(43) Pub. Date: Aug. 27, 2026

(54) MEDICAL ACCESS SYSTEMS AND METHODS

(71) Applicant: INTUITIVE SURGICAL OPERATIONS, INC., Sunnyvale, CA (US)

(72) Inventors: Ryan C. Abbott, San Jose, CA (US); Quirina De Ruiter, Sunnyvale, CA (US)

(21) Appl. No.: 19/542,316

(22) Filed: Feb. 17, 2026

Related U.S. Application Data
(60) Provisional application No. 63/760,312, filed on Feb. 19, 2025.

(51) Int. Cl. A61B 34/30; A61B 1/00

(52) U.S. Cl. CPC A61B 34/30; A61B 1/00045; A61B 1/00094; A61B 1/00148; A61B 1/00158; A61B 2034/301; A61B 2034/305

(57) ABSTRACT

A medical system includes a working port with an outer guide for interfacing with a treatment location, an instrument carriage with an inner guide configured to extend within the outer guide of the working port, and an instrument configured to couple to and be operated via the instrument carriage. The medical system further includes an endoscope and endoscope carriage, where the endoscope and instrument extend through the inner and outer guides to access a treatment location.

### CROSS-REFERENCE TO RELATED APPLICATION

This application claims the benefit of priority to U.S. Provisional Application No. 63/760,312, filed Feb. 19, 2025, which is hereby incorporated by reference herein in its entirety.

### FIELD

Disclosed embodiments relate to robotic medical access systems.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. One such minimally invasive technique is to use a flexible and/or steerable elongate device, such as a flexible catheter, that can be inserted into anatomic passageways and navigated toward a region of interest within the patient anatomy.

### SUMMARY

The following presents a simplified summary of various examples described herein and is not intended to identify key or critical elements or to delineate the scope of the claims.

In accordance with a first example, a medical system is disclosed that includes a working port for interfacing with a treatment location. The working port includes a proximal end configured to rotatably couple to a robotic manipulator arm, a distal end including a beveled tip, the rotatable coupling of the proximal end allowing the beveled distal tip to be rotated at the treatment location, and an outer guide extending from the distal end at least partly to the proximal end. The outer guide defines an outer guide channel including an outer endoscope channel portion, an outer instrument channel portion, and a suction portion, the outer endoscope channel portion configured to receive an endoscope, the outer instrument channel portion configured to receive an instrument. A suction outlet is disposed at the proximal end of the working port, the suction outlet fluidly coupled to the suction portion of the outer guide channel.

In some examples, the medical system can include one or more of the following aspects: the proximal end of the working port includes a basin for liquid collection; the proximal end of the working port defines an opening fluidly coupled to the suction portion for control of pressure within the suction portion; the proximal end includes a magnetic coupling for coupling the working port to the robotic manipulator arm; the outer guide of the working port tapers from the proximal end to the distal end, such that the instrument channel portion is angled relative to the endoscope channel portion; and/or the outer guide has a tapering horizontal cross-section, allowing the endoscope and relatively smaller diameter instrument to extend along one another.

In some examples, the medical system further includes an instrument carriage including one or more actuators and an inner guide configured to extend within the outer guide of the working port, the inner guide defining an inner guide channel including an inner endoscope channel portion for receiving the endoscope and an inner instrument channel portion for receiving the instrument.

In some examples, the instrument carriage is separate from the working port. In further examples, the inner guide defines an irritation channel and, if desired, the inner guide further includes an irrigation inlet port fluidly coupled to irrigation channel.

In some examples, the suction portion of the outer guide channel is defined between the inner guide and the outer guide to define a suction channel and/or the inner guide tapers from a proximal end to a distal end, such that the inner instrument channel portion is angled relative to the inner endoscope channel portion.

In some examples, the instrument carriage and the working port are integrated together. In further examples, the suction portion of the outer guide channel is defined between the inner guide and the outer guide to define a suction channel. The outer guide and the inner guide can further define an irrigation channel therebetween and, if desired, the suction channel can be recessed relative to the irrigation channel. In further examples, the inner guide and the outer guide have a right-cylindrical shape (e.g., have a circular cross-section).

In some examples, the instrument is a first instrument; and the instrument carriage further includes a second instrument channel for a second instrument, the actuators of the instrument carriage including a first set for the first instrument and a second set for the second instrument. In further examples, the medical system further includes the second instrument, the second instrument including an elongate device and a drive unit configured to engage the second set of actuators

In some examples, the medical system further includes an endoscope and an endoscope carriage configured to move the endoscope along an insertion axis and roll the endoscope about the insertion axis.

In some examples, the endoscope and the endoscope carriage are integrated with the instrument carriage. In other examples, the endoscope and the endoscope carriage are separate from the working port and the instrument carriage. In further examples, the medical system includes a releasable locking mechanism configured to secure the endoscope and the instrument carriage together, such that roll of the endoscope about the insertion axis causes the working port and the instrument carriage to roll about the insertion axis.

In some examples, the medical system further includes an instrument including a flexible elongate device and a drive unit having drive inputs configured to engage the one or more actuators of the instrument carriage.

In some examples, the instrument carriage includes at least one actuator and the drive unit comprises at least one drive input to cause insertion of the instrument along the insertion axis. In further examples, the instrument carriage includes at least three actuators and the drive unit comprises at least three drive inputs to impart degrees-of-freedom to the instrument, the degrees-of-freedom including at least three of: insertion along insertion axis, roll about insertion axis, pitch movement, yaw movement, or grasping. In yet further examples, a length of the drive unit along the insertion axis constrains an insertion depth for the instrument, the at least one actuator and the at least one drive input comprise slots and spines for immediate engagement when the instrument is coupled to the instrument carriage, which can provide control of the instrument without homing, and/or the at least one drive input includes at least one capstan.

In some examples, the instrument is configured to releasably couple to the instrument carriage. In further examples, the instrument includes a plurality of interchangeable instruments configured to be releasably coupled to the instrument carriage.

In some examples, the instrument is a kerrisoner including an inner tooth member and outer catheter.

In some examples, the medical system further includes a dilator assembly, the dilator assembly including a plurality of members of increasing diameter to expand an initial incision to provide access for the working port. In further examples, the dilator assembly is configured to provide inner diameter access for the working port or the dilator assembly further includes an outer sleeve to provide outer diameter access for the working port.

In some examples, the medical system further includes a robotic manipulator arm including a plurality of links rotatably coupled together by joints, the robotic manipulator arm configured to operate about a remote center defined relative to the beveled tip of the working port. In further examples, the remote center is between about 2 mm and about 10 mm beyond an access opening in a patient, the endoscope carriage is configured to couple to robotic manipulator arm, such that the robotic manipulator arm controls movement of the endoscope, and/or the instrument carriage includes a housing for the one or more actuators, the housing having beveled longitudinal edges for clearance from the robotic manipulator arm when rotating about the insertion axis.

In some examples, the medical system further includes a display and a control system, wherein the control system is configured to show images from the endoscope with the instrument held at a same location relative to the endoscope even during roll movements.

In further examples, the working port, the instrument carriage, and the endoscope are a first access assembly, and the medical system further includes a second access assembly.

In accordance with a second example, a medical system is disclosed that includes an instrument carriage. The instrument carriage includes a proximal housing including one or more actuators and an inner guide extending distally away from the proximal housing, the inner guide defining an inner endoscope channel configured to receive an endoscope, an inner instrument channel configured to receive an instrument, and an irrigation channel.

In some examples, the proximal housing includes an irrigation inlet port fluidly coupled to the irrigation channel of the inner guide, the inner guide tapers from a proximal end adjacent to the proximal housing to an opposite, distal end, with the inner instrument channel being angled relative to the inner endoscope channel, and/or the inner guide has a tapering horizontal cross-section, allowing the endoscope and relatively smaller diameter instrument to extend along one another.

In some examples, the medical system further includes a working port including: a proximal end configured to rotatably couple to a robotic manipulator arm; a distal end including a beveled tip, the rotatable coupling of the proximal end allowing the beveled distal tip to be rotated at the treatment location; and an outer guide extending from the distal end at least partly to the proximal end and defining an outer guide channel, wherein the inner guide is configured to extend within the outer guide of the working port.

In further examples, the outer guide and the inner guide define a suction channel therebetween, the working port comprising a suction outlet fluidly coupled to the suction channel, the proximal end of the working port includes a basin for liquid collection and defines an opening fluidly coupled to the suction channel for control of pressure within the suction channel, and/or the inner guide and the outer guide have a complementary tapering profile.

In some examples, the medical system further includes an endoscope and an endoscope carriage configured to move the endoscope along an insertion axis and roll the endoscope about the insertion axis.

In some examples, the medical system further includes an instrument including an elongate device and a drive unit having one or more drive inputs configured to engage the one or more actuators of the instrument carriage. In further examples, the one or more actuators and drive inputs includes three or more actuators and drive inputs to impart degrees-of-freedom to the instrument, the degrees-of-freedom including at least three of: insertion along insertion axis, roll about insertion axis, pitch movement, yaw movement, or grasping; a length of the drive unit along the insertion axis constrains an insertion depth for the instrument; and/or the one or more actuators and drive inputs comprise slots and spines for immediate engagement when the instrument is coupled to the instrument carriage.

In accordance with a third aspect, a treatment method is disclosed herein that includes inserting an outer guide of a working port and an inner guide of an instrument carriage through an access opening of a patient to dispose a distal tip of the working port adjacent to a treatment location, the inner guide received within an outer guide channel defined by the outer guide, rotatably coupling the working port to a working port mount of a robotic manipulator arm, inserting an endoscope through an endoscope channel defined by the inner guide to dispose a distal end of the endoscope through the instrument carriage and the working port, and inserting an instrument through an instrument channel defined by the inner guide to dispose a distal end of the instrument at the treatment location distal of the working port.

In some examples, the treatment location is along a spine of the patient and/or the method includes inserting the inner guide of the instrument carriage into the outer guide channel of the outer guide.

In some examples, the method includes irrigating the treatment location through an irrigation channel defined at least partially by the inner guide and applying suction through a suction channel defined at least partially by the outer guide. In further examples, the irrigation channel is defined between the inner guide and the outer guide and/or the method includes partially retracting the instrument along insertion axis to be cleaned adjacent to the suction channel by irrigation flow.

In some examples, the method includes inserting a plurality of dilator members of increasing diameter into the access opening to provide access to the treatment for the working port. In further examples, the method includes inserting the working port over the plurality of dilator members to insert the working port through the access opening; or the method includes inserting an outer sleeve over the plurality of dilator members, removing the plurality of dilator members, and inserting the working port into the outer sleeve to insert the working port through the access opening.

In some examples, inserting the instrument through the instrument channel includes coupling a drive unit of the instrument to the instrument carriage to engage one or more actuators of the instrument carriage; the method includes retracting the instrument from the instrument channel and inserting a second instrument through the instrument channel to dispose a second end effector of the second instrument at the treatment location distal of the working port; the method includes performing a spinal decompression treatment; and/or the working port, the instrument carriage, the robotic manipulator arm, the endoscope, and the instrument are a medical system; and the method includes operating a second medical system to insert a second endoscope through a second access opening for vision of the treatment location from another direction.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a perspective view of a manipulator system according to examples described herein.

FIG. 2A is a perspective view of a first example medical system including a working port, an instrument carriage, an instrument, an endoscope, and an endoscope carriage according to examples described herein.

FIG. 2B is a perspective view of the medical system of FIG. 2A.

FIG. 2C is a cross-sectional view of the working port of the medical system of FIG. 2A.

FIG. 2D is a perspective view of the instrument carriage of the medical system of FIG. 2A.

FIG. 2E is a bottom view of the working port and instrument carriage of the medical system of FIG. 2A.

FIG. 2F is a sectional view of the instrument carriage and instrument of the medical system of FIG. 2A.

FIG. 2G is a sectional view of the instrument carriage and instrument of the medical system of FIG. 2A.

FIG. 2H is a perspective view of a coupling for the instrument carriage and instrument of the medical system of FIG. 2A.

FIG. 2I is a sectional perceptive view of an example instrument for the medical system of FIG. 2A.

FIG. 2J is a perspective view of a second example medical system including a working port, an instrument carriage, an instrument, an endoscope, and an endoscope carriage according to examples described herein.

FIG. 2K is a perspective view of the medical system of FIG. 2J.

FIG. 2L is a cross-sectional view of the instrument carriage and working port of the medical system of FIG. 2J.

FIG. 2M is a bottom view of the working port and instrument carriage of the medical system of FIG. 2J.

FIG. 2N is a perspective view of a dilator system for the medical systems of FIGS. 2A and 2J.

FIG. 3 is a diagrammatic view of a manipulator system including the medical system of FIG. 2A or 2J according to examples described herein.

FIG. 4 is a flowchart illustrating a treatment method according to examples described herein.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

Aspects of this disclosure herein can be part of a computer-assisted teleoperational manipulator system, sometimes referred to as a robotically-assisted manipulator system or a robotic system. The manipulator system can include one or more manipulators that can be operated with the assistance of an electronic controller (e.g., computer) to move and control functions of one or more instruments when coupled to the manipulators.

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees-of-freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

Spinal decompression procedures often require the need to remove bone and/or soft tissue to decompress the spinal cord and/or dorsal nerve roots to reduce back pain, sciatica, and improve quality of life. These procedures face a trade-off between an access opening size and the need to remove healthy bone to gain access to the diseases bone, which can destabilize the spine, as well as removal of muscle and soft tissue, which can impact pain, recovery time, and complications such as infection.

The systems, devices, and methods described herein utilize a working port to provide an endoscope and an instrument access to a treatment location within the spine, as well as irrigation of the treatment location with water. The working port can include a rotatable coupling and a beveled distal tip to allow a user to protect anatomy and push tissue away at the treatment location. In some examples, the working port at least partially provides a channel for irrigation fluid to travel away from a treatment location. Pursuant to this, the working port can include a basin for liquid collection at a proximal end thereof and a suction outlet to ensure that the irrigation fluid stays contained during a procedure.

In some examples, the systems, devices, and methods described herein further include an instrument carriage. The instrument carriage includes one or more actuators configured to drive movement of a coupled instrument and an inner guide configured to extend within the outer guide of the working port. The instrument carriage can be separate from or integrated with the working port. For example, the inner guide can define an irrigation channel to deliver fluid to the treatment location or the inner and outer guides can define the irrigation channel therebetween.

In some examples, the systems, devices, and methods described herein further include an endoscope and an endoscope carriage configured to move the endoscope along an insertion axis and roll the endoscope about the insertion axis. The endoscope and endoscope carriage can be separate from or integrated with the instrument carriage. The endoscope and instrument carriage can also include a releasable locking mechanism that secures endoscope to the instrument carriage, such that roll of the endoscope about the insertion axis causes the instrument carriage, and the working port coupled thereto, to roll about insertion axis.

In some examples, the systems, devices, and methods described herein further include an instrument including an elongate device and a drive unit having drive inputs configured to engage the one or more actuators of the instrument carriage. For example, the drive unit and actuators can be configured to provide three or more degrees-of-freedom for the instrument including insertion along insertion axis, roll about insertion axis, pitch movement, yaw movement, or grasping. Additionally, the drive unit and actuators can include slots and spines for immediate engagement when the instrument is coupled to the instrument carriage. With a releasable coupling, different instruments can be exchanged during a procedure as required.

Aspects of this disclosure herein can be part of a computer-assisted teleoperational manipulator system, sometimes referred to as a robotically-assisted manipulator system or a robotic system. The manipulator system can include one or more manipulators that can be operated with the assistance of an electronic controller (e.g., computer) to move and control functions of one or more instruments when coupled to the manipulators.

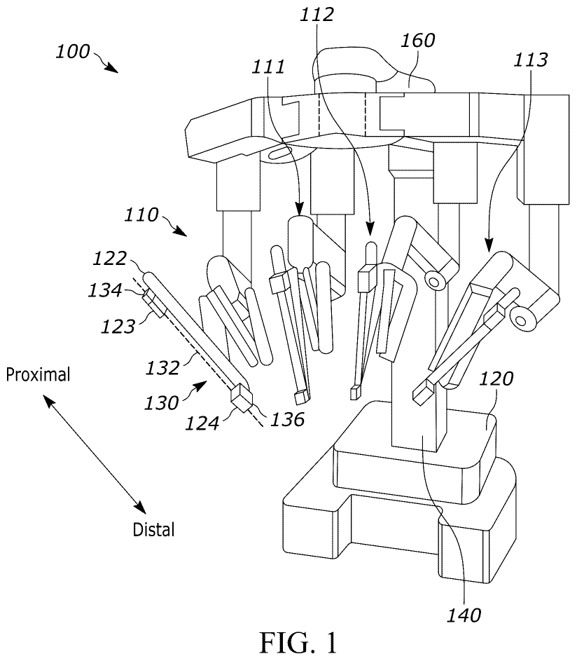

FIG. 1 illustrates an example manipulator system 100. The manipulator system 100 includes a base 120, a main column 140, and a main boom 160 connected to main column 140. Manipulator system 100 also includes a plurality of manipulator arms 110, 111, 112, 113, which are each connected to main boom 160. Manipulator arms 110, 111, 112, 113 each include an instrument mount portion 122 to which an instrument 130 can be mounted, which is illustrated as being attached to manipulator arm 110. While the manipulator system 100 depicts four manipulator arms, various embodiments can include more or fewer manipulator arms.

Instrument mount portion 122 can include a drive assembly 123 and a cannula mount 2124, with a transmission mechanism 134 of the instrument 130 connecting with the drive assembly 123, according to an embodiment. Cannula mount 124 is configured to hold a cannula 136 through which a shaft 132 of instrument 130 can extend to a surgery site during a surgical procedure. Drive assembly 123 contains a variety of drive and other mechanisms that are controlled to respond to input commands at an operator input system and transmit forces to the transmission mechanism 134 to actuate the instrument 130. Although the embodiment of FIG. 1 shows an instrument 130 attached to only manipulator arm 110 for ease of viewing, an instrument can be attached to any and each of manipulator arms 110, 111, 112, 113. For example, the examples described herein can be used with a da Vinci® Surgical System, such as the da Vinci X®, Xi®, or SP® Surgical Systems, all commercialized by Intuitive Surgical, Inc., of Sunnyvale, California.

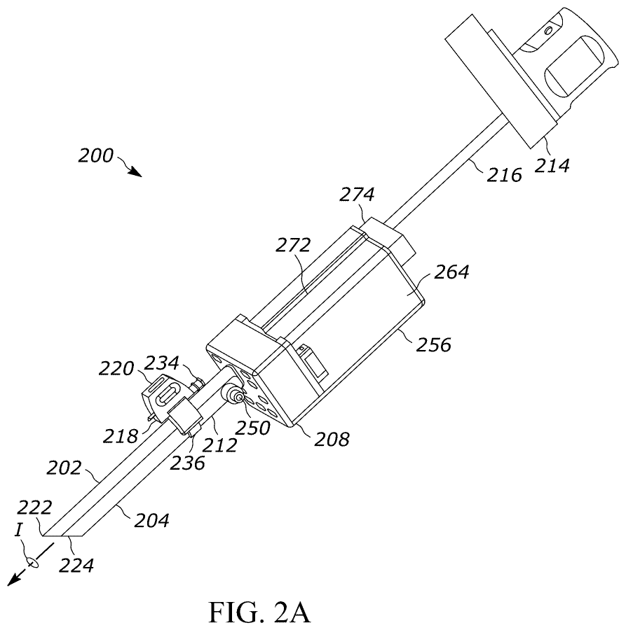

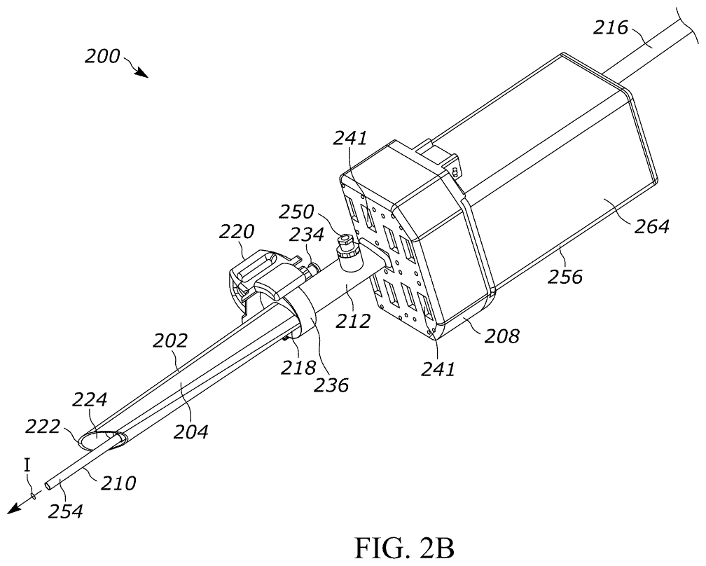

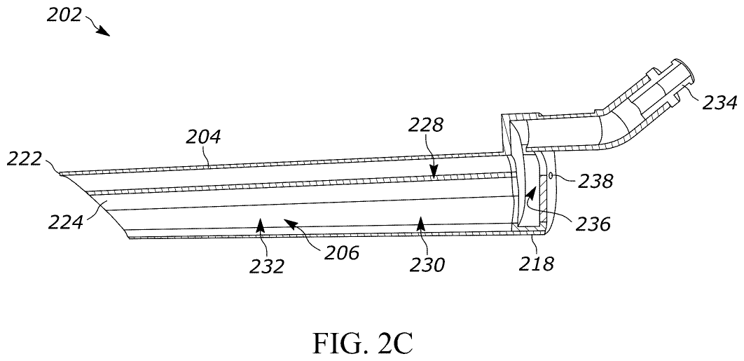

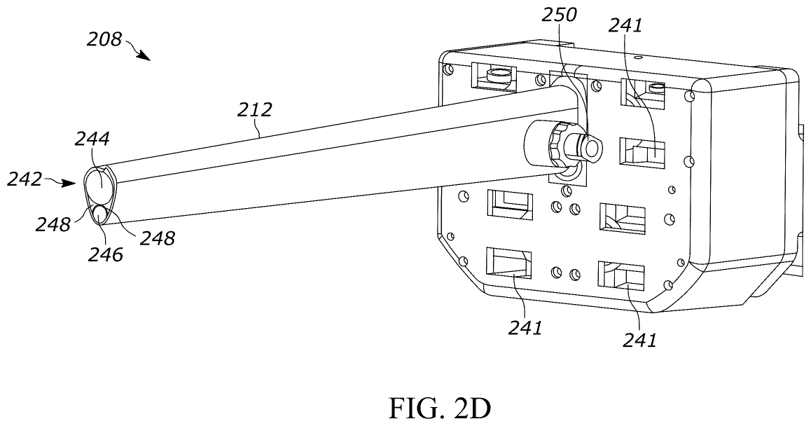

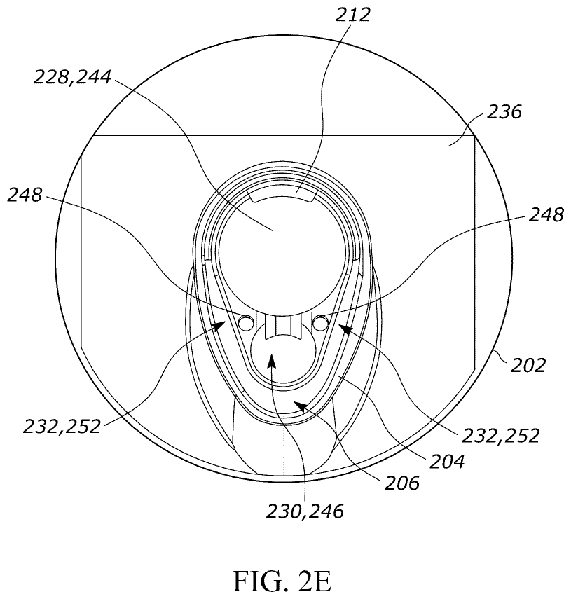

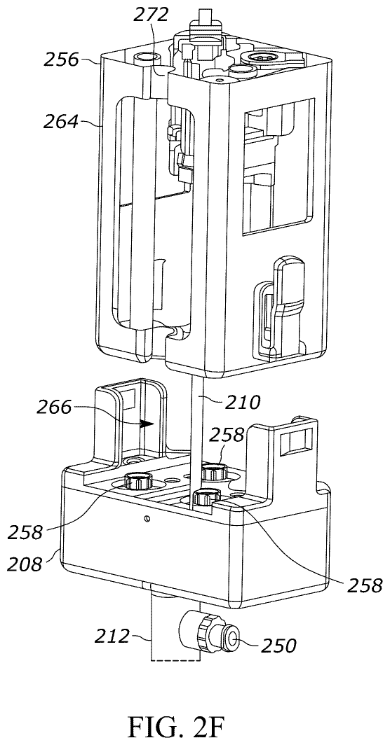

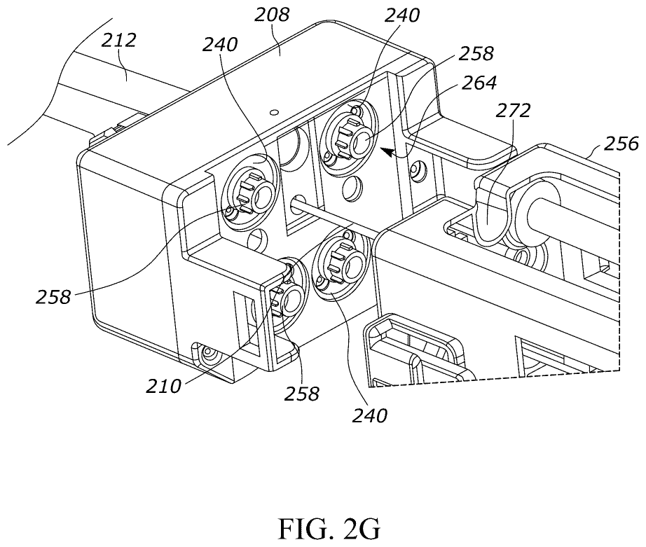

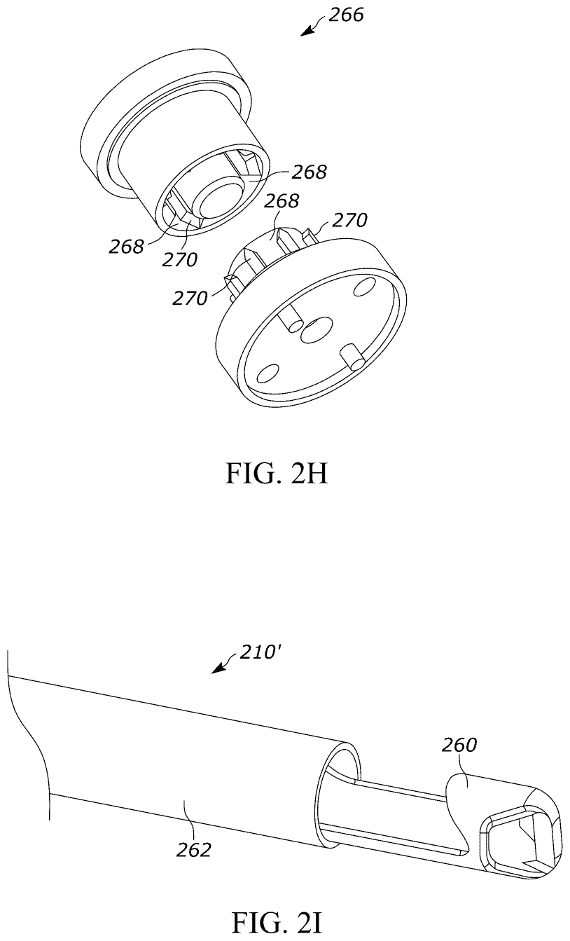

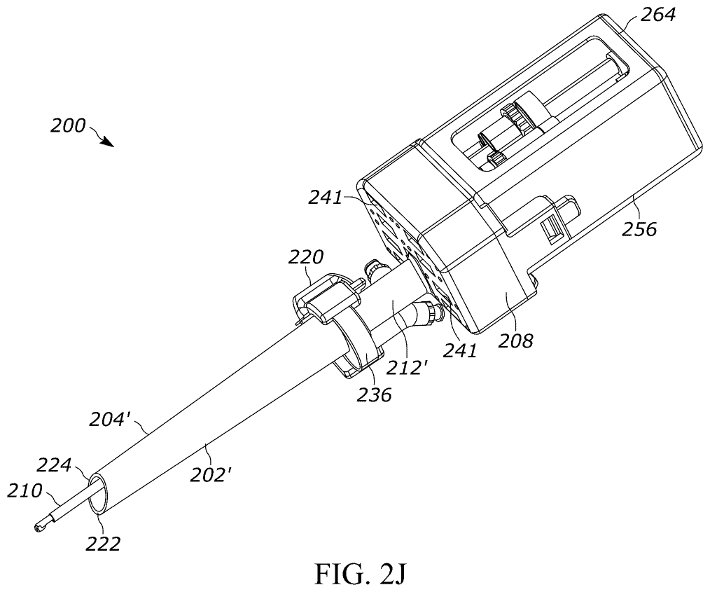

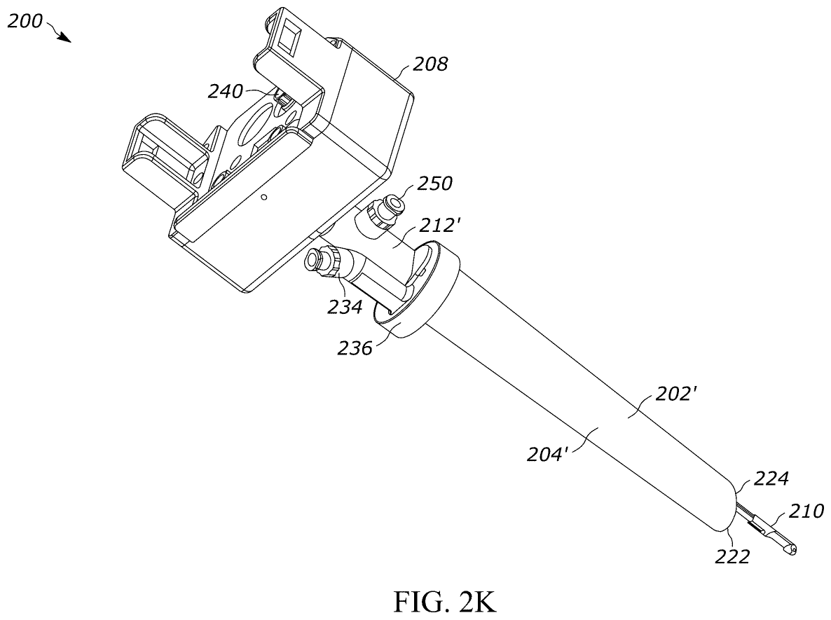

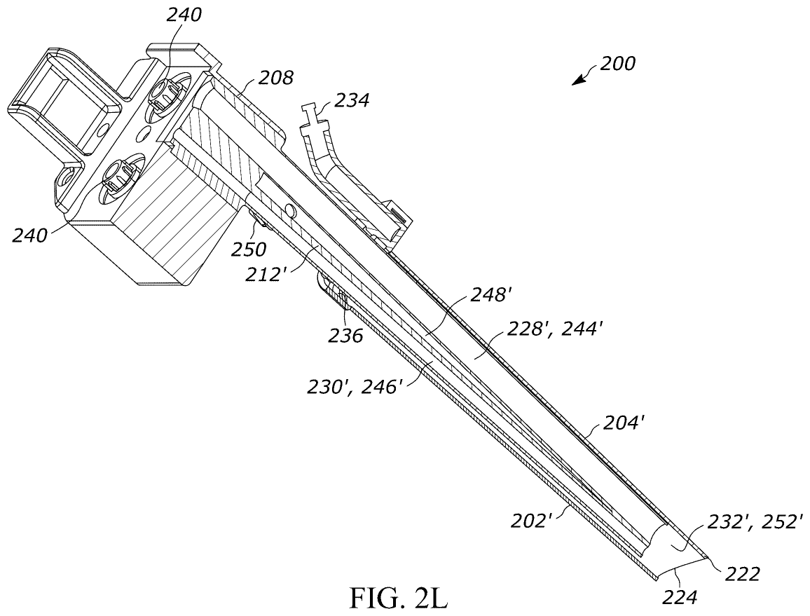

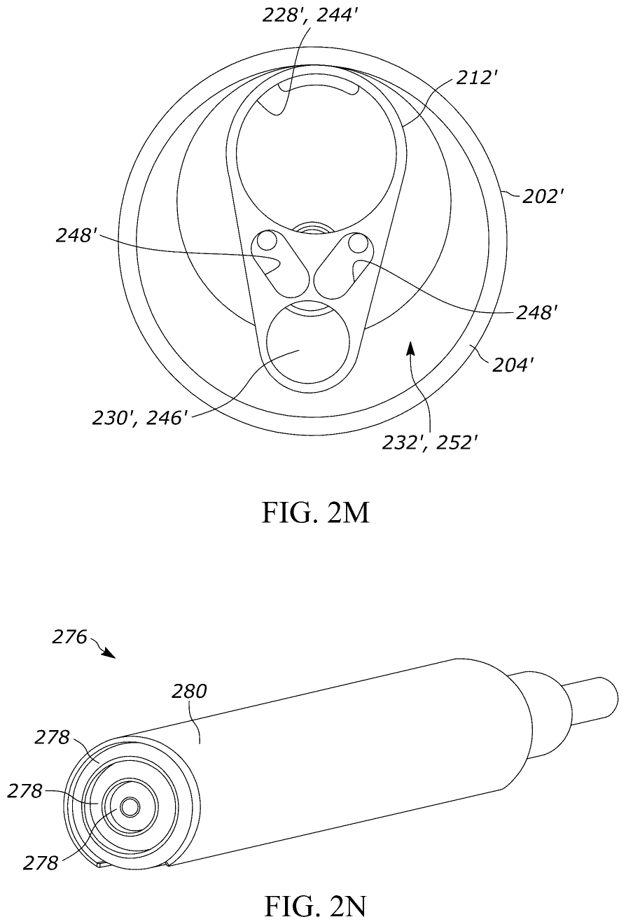

FIGS. 2A-2N show a medical system 200 configured to provide access to a treatment location with a patient, such as within the spinal cord of a patient. The medical system 200 includes a working port 202 for interfacing with the treatment location. The working port 202 includes an outer guide 204 defining an outer guide channel 4206 that provides access to the treatment location through the outer guide 204. The medical system 200 further includes an instrument carriage 208 and an inner guide 212. The instrument carriage 208 is configured to be coupled to an instrument 210 and the inner guide 212, which is coupled to the instrument carriage 208, is configured to be inserted into and extend within the outer guide 204 of the working port 202. The medical system 200 further includes an endoscope carriage 214 configured to couple to an endoscope 216 and move the endoscope 216 along an insertion axis I and roll the endoscope 216 about the insertion axis I. In some examples, the medical system 200 further includes the instrument 210 and the endoscope 216.

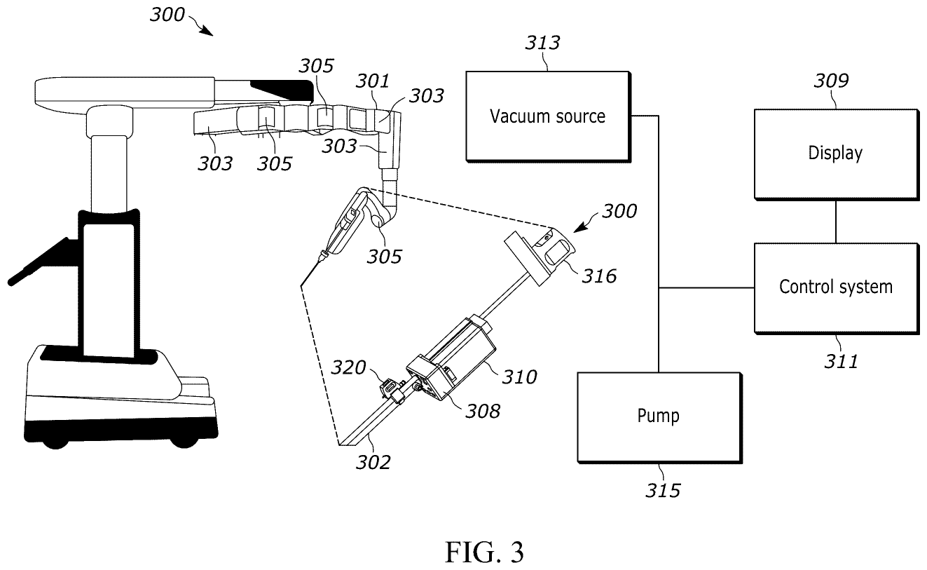

Details of the working port 202, 202′ are shown in FIGS. 2A-2C, 2E, and 2J-2M. The working port 202 includes a proximal end 218 that is configured to rotatably couple to a robotic manipulator arm 301 via a working port mount 220, discussed in more detail below with reference to FIG. 3, and a distal end 222 including a beveled tip 224. The rotatable coupling of the proximal end 218 allows the beveled distal tip 224 to be rotated at the treatment location, which can advantageously be utilized to move or block tissue, nerves, or other objects at the treatment location. In some examples, the proximal end 218 includes part of a magnetic coupling (e.g., a magnet or magnetic material) for coupling the working port 202 to the robotic manipulator arm 301.

As shown, the outer guide 204 extends from the distal end 222 at least partly to the proximal end 218. For example, the outer guide 204 includes the beveled tip 224 and extends to the proximal end 218. The outer guide channel 4206 including an outer endoscope channel portion 228, an outer instrument channel portion 230, and a suction portion 232, where the outer endoscope channel portion 228 is configured to receive the endoscope 216 and the outer instrument channel portion 230 is configured to receive the instrument 210. In one example, the outer guide 204 includes a tubular wall defining the outer guide channel 4206. The outer endoscope channel portion 228, the outer instrument channel portion 230, and the suction portion 232 form portions of the open space within the outer guide 204 defined by the tubular wall.

A suction outlet 234 of the working port 202 is disposed at the proximal end 218 and is fluidly coupled to the suction portion 232 of the outer guide channel 4206. This configuration allows suctioned fluid and/or other materials to be pulled from a treatment location and through the suction outlet 234. In some examples, the proximal end 218 of the working port 202 includes a basin 236 for liquid collection between the suction portion 232 of the outer guide channel 4206 and the suction outlet 234. In the illustrated example, the basin 236 has a cylindrical form and volume extending laterally outwardly from at least a portion of the outer guide 204. As shown, the basin 236 is offset from a longitudinal axis of the outer guide 204. For some procedures, it may be desirable to control an amount of pressure within the suction portion 232. To limit a pressure to a desired level, the proximal end 218 of the working port 202 can define an opening 238 (e.g., to atmosphere) that is fluidly coupled to the suction portion 232.

As shown, the endoscope 216 and the instrument 210 have different diameters, with the endoscope 216 having a relatively larger diameter. Accordingly, with the endoscope 216 and the instrument 210 accommodated in a side-by-side relation within the outer guide 204, the outer guide 204 can have a tapering horizontal cross-section (e.g., a tear drop shape) to minimize the size the outer guide 204 around the diameters of the endoscope 216 and the instrument 210 when the endoscope 216 and the relatively smaller diameter instrument extend along one another within the outer endoscope channel portion 228 and the outer instrument channel portion 230. As shown, the suction portion 232 extends alongside the outer endoscope channel portion 228 and the outer instrument channel portion 230, either along one or both sides thereof.

In some examples, it may be helpful to slightly angle the instrument 210 relative to the endoscope 216, so that the instrument 210 can extend forwardly in front of the endoscope 216 at the treatment location for visibility and ease of work. Accordingly, in these examples, the outer guide 204 tapers (e.g., the outer instrument channel portion 230 is angled inwardly towards a longitudinal axis of the outer guide 204) from the proximal end 218 to the distal end 222, which allows the instrument channel portion 230 to be angled relative to the endoscope channel portion 228.

Details of the instrument carriage 208 and inner guide 212, 212′ are shown in FIGS. 2D-2G and 2K-2M. As shown, the instrument carriage 208 includes one or more actuators 240 configured to drive movement of the instrument 210 along one or more degrees-of-freedom. If applicable, the instrument carriage 208 can define openings 241 therein to provide access to the actuators 240, such as access to sockets or ports of the actuators 240. In some examples, the actuators 240 are servos.

The inner guide 212 extends distally from the instrument carriage 208 and defines an inner guide channel 242 that includes an inner endoscope channel portion 244 for receiving the endoscope 216 and an inner instrument channel portion 246 for receiving the instrument 210. The inner guide 212 is configured to extend within the outer guide 204 of the working port 202, such that when the inner guide 212 is within the outer guide 204, the endoscope 216 extends through the inner endoscope channel portion 244 of the inner guide 212 and the outer endoscope channel portion 228 of the outer guide 204 and the instrument 210 extends through the inner instrument channel portion 246 of the inner guide 212 and the outer instrument channel portion 230 of the outer guide 204.

In some examples, the inner endoscope channel portion 244 and the inner instrument channel portion 246 are discrete throughbores extending within the inner guide 212, such that the endoscope 216 and the instrument 210 are separated by the inner guide 212. In other examples, the inner endoscope channel portion 244 and the inner instrument channel portion 246 are at least partially connected within the inner guide 212.

The instrument carriage 208 and the inner guide 212 can be permanently coupled together to form a single component for the medical system 200. In some alternative examples, the instrument carriage 208 and the inner guide 212 can be configured to releasably couple together by any suitable mechanism, such as snap-fit, luer connectors, latches, and so forth.

In a first example as shown in FIGS. 2A-2E, the instrument carriage 208 is separate from the working port 202. As discussed above, due to the differing diameters of the endoscope 216 and the instrument 210, the inner guide 212 can have a tapering horizontal cross-section (e.g., a tear drop shape) similar to the outer guide 204 to minimize the size the inner guide 212 around the diameters of the endoscope 216 and the instrument 210 when the endoscope 216 and the relatively smaller diameter instrument extend along one another within the inner guide 212. As shown, the suction portion 232 extends alongside the outer endoscope channel portion 228 and the outer instrument channel portion 230, either along one or both sides thereof.

Further, in the examples where is it helpful to slightly angle the instrument 210 relative to the endoscope 216, the inner guide 212 tapers (e.g., the inner instrument channel portion 246 is angled inwardly towards a longitudinal axis of the inner guide 212) from the instrument carriage 208 to a distal end of the inner guide 212, which allows the inner instrument channel portion 246 to be angled relative to the inner endoscope channel portion 244.

For procedures where irrigation is helpful, the inner guide 212 defines an irritation channel 248 to deliver irrigation fluid to the treatment location. The irrigation channel 248 extends along the inner endoscope channel portion 244 and the inner instrument channel portion 246 within the inner guide 212 with an outlet at a distal end of the inner guide 212. In some examples, the inner guide 212 defines a plurality of irrigation channels 248 to accommodate spacing within the inner guide 212 and deliver a desired amount of irrigation fluid to the treatment location. Further, the inner guide 212 includes an irrigation inlet port 250 fluidly coupled to the irrigation channel 248 to supply irrigation fluid thereto. As discussed above, the outer guide channel 4206 includes a suction portion 232 for suctioning irrigation fluid from the treatment location. The suction portion 232 can extend along an outer surface of the inner guide 212, such that the inner guide 212 and the outer guide 204 define a suction channel 252 therebetween.

Another example is shown in FIGS. 2J-2M. In this example, the inner guide 212′ and the working port 202′ are integrated together and fixedly coupled to the instrument carriage 208. As shown, similar features between the examples have similar reference characters. As such, the description of features in the above example included in this example are equally applicable. Differences between the examples will be described below.

In this example, the outer endoscope channel portion 228′ and the inner endoscope channel portion 244′ are defined by the same throughbore and the outer instrument channel portion 230′ and the inner instrument channel portion 246′ are defined by the same throughbore. The throughbores can be discrete or at least partially overlapping. Further, the suction portion 232′ of the outer guide channel 4206′ is defined between the inner guide 212′ and the outer guide 204′ to define the suction channel 252′. The outer guide 204′ and the inner guide 212′ also define the irrigation channel 248′ therebetween.

In some examples, the outlet of the suction channel 252′ is recessed relative to outlet of the irrigation channel 248′. This configuration allows a user to partially retract the instrument 210 to place an end of the instrument (e.g., an end effector or other tool) within a flow path between the irrigation channel 248′ and the suction channel 252′ to effectively clean the end of the instrument in situ without having to fully retract the instrument 210 during a procedure.

By integrating the inner guide 212′ and the working port 202′ together, the inner guide 212′ and the outer guide 204 can have a generally right-cylindrical shape, such as with a circular cross-section. In the examples where it is helpful to slightly angle the instrument 210 relative to the endoscope 216, the outer and inner instrument channel portions 230′, 246′ can be tapered (e.g., angled inwardly towards a longitudinal axis of the inner guide 212′/working port 202′), which allows the instrument channel portions 230′, 246′ to be angled relative to the outer and inner endoscope channel portions 228′, 244′.

Further details of the instrument carriage 208 are described with reference to FIGS. 2D-2G and 2K-2M in combination with details of the instrument 210. The instrument 210 includes a flexible elongate device 254 and a drive unit 256 having drive inputs 258 configured to engage the one or more actuators 240 of the instrument carriage 208. In one example, one or more of the drive inputs 258 can be capstans to control movement of an articulable body portion of the flexible elongate device 254 via tendons or pull wires.

In some examples, the actuators 240 and the drive inputs 258 include one, two, or at least three pairs that impart degrees-of-freedom to the instrument 210, including, for example, movement of the instrument 210, as well as movement or actuation of an end effector of the instrument 210. Of course, additional actuators 240 and drive inputs 258 can be provided to impart four, five, six, or more degrees-of-freedom to the instrument 210. The degrees-of-freedom for the instrument can include one or more, or at least three of: insertion along the insertion axis I, roll about insertion axis I, pitch movement, yaw movement, or grasping. In an additional example, the instrument 210 can be a kerrisoner 210′ that includes an inner tooth member 260 and an outer catheter 262. One of the degrees-of-freedom for the kerrisoner 210′ includes moving one of the inner tooth member 260 or the outer catheter 262 towards the other to cause the inner tooth member 260 to cut a portion of tissue or other material for sampling.

The drive unit 256 includes a housing 264 containing the drive inputs 258. As can be appreciated, the depth of the housing 264 along the direction of the insertion axis I constrains or defines an available length of the instrument 210 to be driven along the insertion axis I and, as such, constrains/defines an insertion depth for the instrument 210. In some examples, an instrument 210 may be sufficiently flexible to coil or wind within the housing 264 to provide additional insertion depth.

One example engagement configuration for the actuators 240 and drive inputs 258 is shown in FIG. 2H. As shown, the actuators 240 and drive inputs 258 include couplings 266 having slots 268 and spines 270 that engage one another when the drive unit 256 is coupled to the instrument carriage 208. The spines 270 can have pointed distal ends to guide the spines 270 to the slots 268 during coupling. Further, one of the couplings 266 can be a plug coupling within the slots 268 and spines 270 defines along an outwardly facing surface and the other of the couplings 266 can be a socket coupling having an outer wall defining the slots 268 and spines 270 on an inwardly facing surface to receive the plug coupling therein. Due to the above configuration, the slots 268 and spines 270 provide an immediate engagement when the instrument 210 is coupled to the instrument carriage 208, which can advantageously provide control of the instrument 210 without a homing action as required with other coupling types.

As discussed, the instrument 210 can be configured to releasably couple to the instrument carriage 208. Securing the instrument 210 to the instrument carriage 208 can be done by any suitable mechanism, including a latch as shown, a pressure fit, snap fit, a fastener, and so forth. Furthermore, because the instrument 210 is releasable, the medical system 200 can include a plurality of different instruments 210 that can be interchanged between or during a procedure to provide a user with a desired functionality at the treatment location. The instrument types can include, for example, a kerrisoner, forceps or other type of grasping device, a camera, an energy treatment device, and so forth.

Although the above examples includes a single instrument 210 and associated channel portions 230, 246, the instrument carriage 208 can be configured to be coupled to two instruments 210 in a side-by-side relation. In these examples, the instrument carriage 208 includes a first set of actuators 240 for the first instrument 210 and a second set of actuators 240 for the second instrument 210. Further, the outer guide 204 includes two outer instrument channel portions 230 and the inner guide 212 includes two inner instrument channel portions 246 that extend along the endoscope channel portions 228, 244. With this configuration, the two instruments 210 can be coupled to the instrument carriage 208 to engage the drive units 256 therewith and insert the flexible elongate devices 254 into the channel portions 230, 246.

As shown in FIG. 2A, the endoscope carriage 214 is positioned to drive the endoscope alongside the instrument carriage 208 and instrument 210 to be inserted into and through the endoscope channel portions 228, 244. In one example, the endoscope 216 and the endoscope carriage 214 are separate from the working port 202 and the instrument carriage 208. For example, the endoscope carriage 214 can be mounted to and movable by the robotic manipulator arm 301 and the working port 202 can have a separate coupling to the robotic manipulator arm 301 to position the components relative to one another and an access opening to a patient. Further, the instrument carriage 208 of this example defines a groove or channel 272 for the endoscope 216 to be inserted therethrough and access the outer and inner endoscope channel portions 228, 244.

As discussed above, the endoscope carriage 214 is configured to roll the endoscope 216 about the insertion axis I. In some examples, the medical system 200 includes a releasable locking mechanism 274 that is configured to secure the endoscope 216 and the instrument carriage 208 together, such that roll of the endoscope 216 about the insertion axis I also causes the instrument carriage 208 and the working port 202 to roll about the insertion axis I. With this configuration, a user can utilize the roll of the endoscope 216 to maneuver and position the beveled tip 224 of the outer guide 204 at the treatment location.

In another example, the endoscope carriage 214 and the endoscope 216 can be integrated with the instrument carriage 208, with endoscope carriage 214 including a drive (e.g., a servo) to drive movement of the endoscope 216 along and about the insertion axis I.

As shown in FIG. 2N, the medical system 200 can include a dilator assembly 276 that is utilized to expand an access opening in a patient to a size sufficient to insert the outer guide 204 therethrough. The dilator assembly 276 includes a plurality of dilator members 278 of increasing diameter, such that the dilator members 278 can be sequentially inserted into the access opening to expand an initial incision to a sufficient size to provide access for the working port 202. In a first approach, the working port 202 and the dilator assembly 276 can be configured so that the outer guide 202 fits over the largest dilator member 278 and is inserted into the access opening around the largest dilator member 278 (e.g., the dilator assembly 276 provides inner diameter access for the working port 202). In a second approach, the dilator assembly 276 further includes an outer sleeve 280 that fits over the largest dilator 278 and is sized so that the outer guide 204 fits therethrough (e.g., the dilator assembly 276 provides outer diameter access for the working port 202).

A manipulator system 300 including the medical system 200 discussed above is shown schematically in FIG. 3. The system 300 includes a robotic manipulator arm 301 including a plurality of links 303 rotatably coupled together by joints 305. In examples consistent with the above disclosure, the manipulator system 300 can correspond to the manipulator system 100.

The robotic manipulator arm 301 is configured to operate about a remote center defined relative to the distal beveled tip 224 of the working port 202. In some examples, the remote center is between about 2 mm and about 10 mm beyond an access opening in a patient. The endoscope carriage 214 couples to the robotic manipulator arm 301 and is driven thereby, such that the robotic manipulator arm 301 controls movement of the endoscope 216 (e.g., insertion along the insertion axis I and roll about the insertion axis I).

Further, the robotic manipulator arm 301 includes the working port mount 220 configured to have the working port 202 coupled thereto during a procedure. The working port mount 220 and working port 202 can have a magnetic coupling therebetween, as discussed above. The working port mount 220 registers the working port 202 with the robotic manipulator arm 301 so that the remote center is accurately reflected in the position of the beveled tip 224.

In examples where the instrument carriage 208 is rotated about the insertion axis I, such as via the operation of the endoscope 216, manual rotation, and so forth, the housing 264 of the instrument carriage 208 can include beveled longitudinal edges 265 for clearance from the robotic manipulator arm 301 when rotating about the insertion axis I.

As shown, the manipulator system 300 further includes a display 309 and a control system 311. With this configuration, the control system 311 is configured to show images from the endoscope 216 during a procedure. Advantageously, the control system 311 can be configured to stabilize or hold the relative positions of the endoscope 216 and instrument 210 on the display 309 regardless of roll movements (e.g., the instrument 210 is always shown at one side of the display 309, such as the bottom or top of the display 309). With this configuration, the user is shown the instrument 210 at a same location relative to the endoscope 216 even during roll movements.

In some examples, the manipulator system 300 further includes a vacuum source 313 for being fluidly coupled to the suction outlet 234 and applying suction through the suction portion 232/suction channel 252, as well as a pump 515 for being fluidly coupled to the irrigation inlet port 250 for supplying irrigation fluid through the irrigation channel 248 to the treatment location.

Although a single medical system 200 is shown in FIG. 3, one or more additional medical systems 200 can be utilized to provide access to the treatment location or another adjacent treatment location during a procedure. For example, two medical system 200 can be utilized for translaminar access on opposite sides of the spine, with both systems 200 providing images of the treatment location from different perspectives. The medical systems 200 of this example can include any or all of the above components and functionalities.

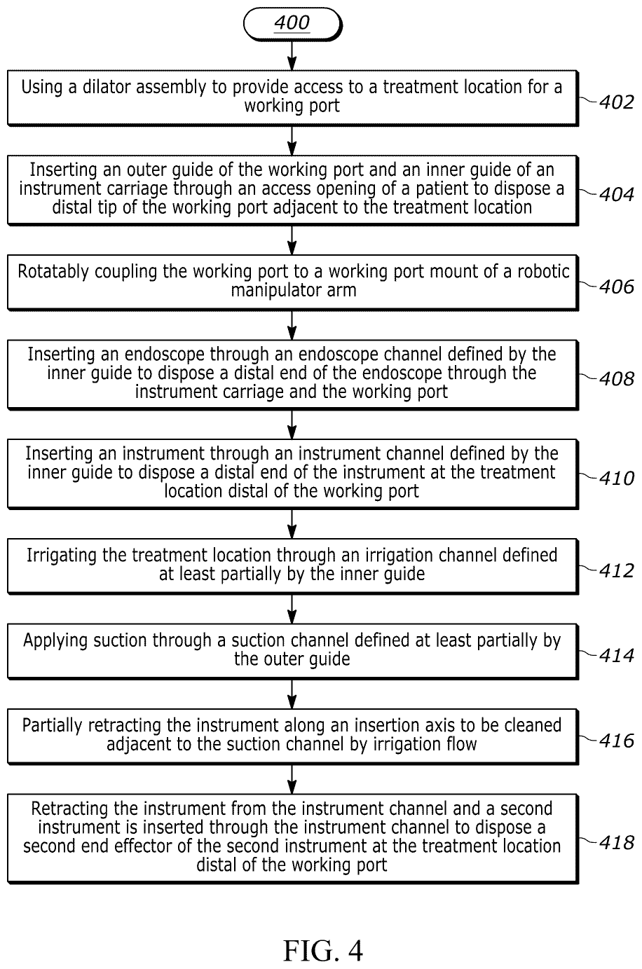

FIG. 4 illustrates a treatment method 400 for a medical system (e.g., medical system 200, manipulator system 300) according to some embodiments. In one example, a treatment location for the method 400 can be along a spine of a patient. In one example, the treatment method 400 is to perform a spinal decompression treatment. The method 400 is illustrated as a set of operations or processes 402 through 420. Not all of the illustrated processes may be performed in all embodiments of method 400. Additionally, one or more processes that are not expressly illustrated in FIG. 4 may be included before, after, in between, or as part of the processes 402 through 420. Processes may also be performed in different orders. In some embodiments, one or more of the processes 402 through 420 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 402 through 420 may be performed by a controller (e.g., control system 311).

In process 402, a dilator assembly (e.g., dilator assembly 276) is utilized to provide access to a treatment location for a working port (e.g., working port 202). Process 402 can include, for example, inserting a plurality of dilator members (e.g., dilator members 278) of increasing diameter into the access opening to provide access to the treatment for the working port. Further, process 402 can include inserting the working port over the plurality of dilator members to insert the working port through the access opening or inserting an outer sleeve (e.g., outer sleeve 280) over the plurality of dilator members. Additionally, process 402 can include removing the plurality of dilator members and inserting the working port into the outer sleeve to insert the working port through the access opening.

In process 404, an outer guide (e.g., outer guide 204) of the working port and an inner guide (e.g., inner guide 212) of an instrument carriage (e.g., instrument carriage 208) are inserted through an access opening of a patient to dispose a distal tip (e.g., distal end 222) of the working port adjacent to the treatment location. In this configuration, the inner guide is received (e.g., inserted) within an outer guide channel (e.g., outer guide channel 4206) defined by the outer guide. In process 406, the working port is rotatably coupled to a working port mount (e.g., working port mount 220) of a robotic manipulator arm (e.g., robotic manipulator arm 301).

In process 408, an endoscope (e.g., endoscope 216) is inserted through an endoscope channel (e.g., endoscope channel 244) defined by the inner guide to dispose a distal end of the endoscope through the instrument carriage and the working port. In process 410, an instrument (e.g., instrument 210) is inserted through an instrument channel (e.g., instrument channel 246) defined by the inner guide to dispose a distal end (e.g., an end effector) of the instrument at the treatment location distal of the working port. In one example, inserting the instrument through the instrument channel includes coupling a drive unit (e.g., drive unit 256) of the instrument to the instrument carriage to engage one or more actuators (e.g., actuators 240) of the instrument carriage.

In process 412, the treatment location is irrigated through an irrigation channel (e.g., irrigation channel 248) defined at least partially by the inner guide and, in process 414, suction is applied through a suction channel (e.g., suction channel 252) defined at least partially by the outer guide. In process 416, the instrument is partially retracted along an insertion axis to be cleaned adjacent to the suction channel by irrigation flow.

In process 418, the instrument is retracted from the instrument channel and a second instrument is inserted through the instrument channel to dispose a second end effector of the second instrument at the treatment location distal of the working port.

In some examples, the working port, the instrument carriage, the robotic manipulator arm, the endoscope, and the instrument are one system and the method 400 further includes operating a second system (e.g., medical system 200, manipulator system 300) to insert a second endoscope through a second access opening for vision of the treatment location from another direction. The second system can further include a second instrument, second working port, second instrument and instrument carriage, and so forth.

One or more components of the embodiments discussed in this disclosure, such as control system 311, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

1. A medical system comprising:
a working port for interfacing with a treatment location, the working port comprising:
a proximal end configured to rotatably couple to a robotic manipulator arm;
a distal end including a beveled tip, the rotatable coupling of the proximal end allowing the beveled distal tip to be rotated at the treatment location;
an outer guide extending from the distal end at least partly to the proximal end, the outer guide defining an outer guide channel including an outer endoscope channel portion, an outer instrument channel portion, and a suction portion, the outer endoscope channel portion configured to receive an endoscope, the outer instrument channel portion configured to receive an instrument; and
a suction outlet disposed at the proximal end of the working port, the suction outlet fluidly coupled to the suction portion of the outer guide channel.

2. The medical system of claim 1, wherein the proximal end of the working port comprises one or more of:
a basin for liquid collection; or
a magnetic coupling for coupling the working port to the robotic manipulator arm.

3. The medical system of claim 1, wherein the proximal end of the working port defines an opening fluidly coupled to the suction portion for control of pressure within the suction portion.

4. (canceled)

5. The medical system of claim 1, wherein the outer guide of the working port at least one of:
tapers from the proximal end to the distal end, such that the instrument channel portion is angled relative to the endoscope channel portion; or
has a tapering horizontal cross-section, allowing the endoscope and relatively smaller diameter instrument to extend along one another.

6. (canceled)

7. The medical system of claim 1, further comprising:
an instrument carriage including one or more actuators; and
an inner guide configured to extend within the outer guide of the working port, the inner guide defining an inner guide channel including an inner endoscope channel portion for receiving the endoscope and an inner instrument channel portion for receiving the instrument.

8. The medical system of claim 7, wherein the instrument carriage is separate from the working port.

9. The medical system of claim 8, wherein the inner guide defines an irritation channel; and further comprises an irrigation inlet port fluidly coupled to the irrigation channel.

10. (canceled)

11. The medical system of claim 7, wherein the suction portion of the outer guide channel is defined between the inner guide and the outer guide to define a suction channel.

12. The medical system of claim 7, wherein the inner guide tapers from a proximal end to a distal end, such that the inner instrument channel portion is angled relative to the inner endoscope channel portion.

13. The medical system of claim 7, wherein the instrument carriage and the working port are integrated together; and the suction portion of the outer guide channel is defined between the inner guide and the outer guide to define a suction channel; and the outer guide and the inner guide further define an irrigation channel therebetween.

14.-15. (canceled)

16. The medical system of claim 13, wherein the suction channel is recessed relative to the irrigation channel.

17. -18. (Canceled)

19. The medical system of claim 7, wherein the instrument comprises a first instrument; and the instrument carriage further comprises a second instrument channel for a second instrument, the actuators of the instrument carriage including a first set for the first instrument and a second set for the second instrument.

20. The medical system of claim 19, further comprising the second instrument, the second instrument including an elongate device and a drive unit configured to engage the second set of actuators.

21. The medical system of claim 7, further comprising:
an endoscope; and
an endoscope carriage configured to move the endoscope along an insertion axis and roll the endoscope about the insertion axis.

22. (canceled)

23. The medical system of claim 21, wherein the endoscope and the endoscope carriage are separate from the working port and the instrument carriage.

24. The medical system of claim 23, further comprising a releasable locking mechanism configured to secure the endoscope and the instrument carriage together, such that roll of the endoscope about the insertion axis causes the working port and the instrument carriage to roll about the insertion axis.

25. The medical system of claim 21, further comprising an instrument including a flexible elongate device and a drive unit having drive inputs configured to engage the one or more actuators of the instrument carriage; and wherein the instrument carriage comprises at least one actuator and the drive unit comprises at least one drive input to cause insertion of the instrument along the insertion axis.

26.-27. (canceled)

28. The medical system of claim 25, wherein a length of the drive unit along the insertion axis constrains an insertion depth for the instrument.

29.-37. (canceled)

38. The medical system of claim 21, further comprising a robotic manipulator arm including a plurality of links rotatably coupled together by joints, the robotic manipulator arm configured to operate about a remote center defined relative to the beveled tip of the working port; wherein the endoscope carriage is configured to couple to the robotic manipulator arm, such that the robotic manipulator arm controls movement of the endoscope.

39.-41. (canceled)

42. The medical system of claim 21, further comprising:
a display; and
a control system, wherein the control system is configured to show images from the endoscope with the instrument held at a same location relative to the endoscope even during roll movements.

43.-69. (canceled)
